(19) United States
(12) Patent Application Publication
(10) Pub. No.: US 2024/0138806 A1
(43) Pub. Date: May 2, 2024

(54) VASCULAR PUNCTURE DEVICE AND VASCULAR PUNCTURE SYSTEM

(71) Applicant: TERUMO KABUSHIKI KAISHA, Tokyo (JP)

(72) Inventors: Takito INUKAI, Isehara-shi (JP); Takumi FUKUDA, Hadano-shi (JP); Yoichiro KUWANO, Tokyo (JP)

(73) Assignee: TERUMO KABUSHIKI KAISHA, Tokyo (JP)

(21) Appl. No.: 18/408,726

(22) Filed: Jan. 10, 2024

Related U.S. Application Data
(63) Continuation of application No. PCT/JP2022/027825, filed on Jul. 15, 2022.

(30) Foreign Application Priority Data
Jul. 21, 2021 (JP) 2021-120218

(51) Int. Cl. A61B 8/08; A61B 17/34; G06T 7/00

(52) U.S. Cl. CPC A61B 8/085; A61B 17/3403; G06T 7/00; G06T 2207/30101

(57) ABSTRACT

A vascular puncture device is a vascular puncture device that punctures a blood vessel using an imaging unit that comes into contact with a skin surface and acquires a cross-sectional image of a human body, a puncture unit including a sharp needle tip, and a drive unit moving the puncture unit, and includes a control unit that can receive information on the cross-sectional image and control an operation of the drive unit. The control unit identifies at least one blood vessel from the information on the cross-sectional image, determines whether the blood vessel is an artery or a vein from the information on the cross-sectional image of the identified blood vessel, sets a blood vessel to be punctured from among a plurality of the identified blood vessels, and controls the drive unit to move the puncture unit to puncture the blood vessel set to be punctured with the puncture unit.

### CROSS-REFERENCES TO RELATED APPLICATIONS

This application is a continuation of International Application No. PCT/JP2022/027825 filed on Jul. 15, 2022, which claims priority to Japanese Application No. 2021-120218 filed on Jul. 21, 2021, the entire content of both of which is incorporated herein by reference.

### TECHNOLOGICAL FIELD

The present invention relates to a vascular puncture device and a vascular puncture system capable of detecting and puncturing a position of a blood vessel from an image acquired by an echographic device.

### BACKGROUND DISCUSSION

In order to secure an access position with respect to a blood vessel for drug administration and endovascular treatment, vascular puncture for puncturing a human body with an injection needle is performed. In the vascular puncture, an operator cannot visually observe the blood vessel from a skin surface, and thus, a position of the blood vessel is estimated by standard knowledge of blood vessel running and skill such as tactile perception of blood vessel pulsation. However, erroneous puncture of the blood vessel often occurs, which causes physical and mental distress to a patient.

For the purpose of reducing the erroneous puncture, in recent years, there is a device that identifies a position of a blood vessel by a sensor, determines a puncture angle and a puncture path from a shape of the blood vessel or the like, and automatically performs vascular puncture by a robot arm (see, for example, U.S. Pat. No. 9,364,171).

However, the device described in U.S. Pat. No. 9,364,171 cannot solve a problem of puncturing an unintended blood vessel by mistaking an artery and a vein which are parallel from a skin surface.

### SUMMARY

A vascular puncture device and a vascular puncture system are disclosed, which are capable of suppressing erroneous puncture of an unintended blood vessel and puncturing a blood vessel with relatively high positional accuracy regardless of skill of an operator.

A vascular puncture device is disclosed that punctures a blood vessel using an imaging unit that comes into contact with a skin surface and acquires a cross-sectional image of a human body, a puncture unit including a needle tip that is sharp, and a drive unit that moves the puncture unit, and includes a control unit that is capable of receiving information on the cross-sectional image and controls an operation of the drive unit. The control unit identifies at least one blood vessel from information on the cross-sectional image, determines whether the at least one blood vessel is an artery or a vein from information on the cross-sectional image of the identified at least one blood vessel, sets a blood vessel to be punctured from among the identified at least one blood vessel, and controls the drive unit to move the puncture unit to puncture the blood vessel set to be punctured with the puncture unit.

A vascular puncture system is disclosed that includes: an imaging unit that comes into contact with a skin surface and acquires a cross-sectional image of a human body; a puncture unit including a needle tip that is sharp; a drive unit that moves the puncture unit; and a control unit that is capable of receiving information on the cross-sectional image and controls an operation of the drive unit. The control unit identifies at least one blood vessel from the information on the cross-sectional image, determines whether the identified at least one blood vessel is an artery or vein from information on the cross-sectional image of the identified at least one blood vessel, sets a blood vessel to be punctured from among the identified at least one blood vessel, and controls the drive unit to move the puncture unit to puncture the blood vessel set to be punctured with the puncture unit.

A method is disclosed for puncturing a blood vessel, the method comprising: acquiring a cross-sectional image of a human body; identifying one or more blood vessels from the acquired cross-sectional image of the human body; determining whether the one or more blood vessels is an artery or a vein from the cross-sectional image of the human body; setting a blood vessel to be punctured from among the one or more identified blood vessels; and puncturing the blood vessel set to be punctured.

The vascular puncture device and the vascular puncture system configured as described above can automatically detect and puncture a blood vessel, and thus, can help suppress the erroneous puncture of an unintended blood vessel and puncture the blood vessel with the high positional accuracy regardless of the skill of the operator.

### BRIEF DESCRIPTION OF THE DRAWINGS

FIG. 1 is a side view of a vascular puncture system according to the present embodiment.

FIG. 2 is a top view of the vascular puncture system, which illustrates a positional relationship with an arm whose cross-sectional image is to be acquired.

FIG. 3 is a configuration diagram of the vascular puncture system.

FIG. 4 is a view illustrating an example of an image acquired by an imaging unit.

FIG. 5 is a side view illustrating the vascular puncture system immediately before puncture in a state where a probe is inclined with respect to a skin surface.

FIG. 6 is a top view illustrating the vascular puncture system immediately before the puncture in the state where the probe is inclined with respect to the skin surface.

FIG. 7 is a side view illustrating the vascular puncture system immediately after the puncture in the state where the probe is inclined with respect to the skin surface.

FIG. 8 is a flowchart illustrating a flow of control in a control unit.

### DETAILED DESCRIPTION

Set forth below with reference to the accompanying drawings is a detailed description of embodiments of a vascular puncture device and a vascular puncture system capable of detecting and puncturing a position of a blood vessel from an image acquired by an echographic device. Note that dimensional ratios in the drawings may be exaggerated and different from actual ratios for convenience of description.

A vascular puncture system 10 according to the embodiment of the present can be used when puncturing an arm H of a human body to acquire a cross-sectional image of the arm H, detect a position of an artery to be punctured, and automatically puncture the artery.

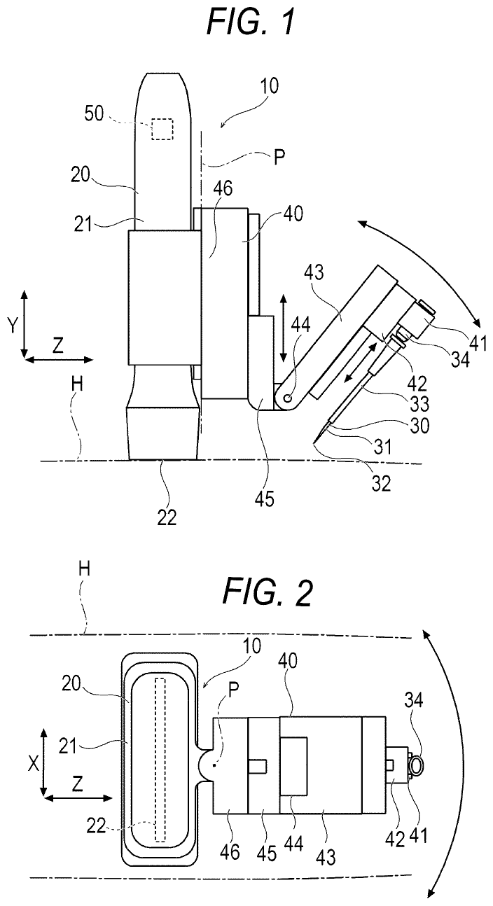

As illustrated in FIG. 1 to 3, the vascular puncture system 10 includes: a probe 20 having an imaging unit 22 that comes into contact with a skin surface and acquires a cross-sectional image of a human body; a puncture unit 30 that performs puncture; a drive unit 40 that moves the puncture unit 30 with respect to the probe 20; an inclination detection unit 50 that detects an inclination angle of the probe 20; and a vascular puncture device 11. The vascular puncture device 11 includes a control unit 60 that performs image analysis of the cross-sectional image and controls the drive unit 40.

The probe 20 includes a vertically elongated handle portion 21 gripped by an operator, an imaging unit 22 disposed at a lower end of the handle portion 21, a transmitter 23 that transmits a signal from the control unit 60 to the imaging unit 22, and a receiver 24 that transmits a signal from the imaging unit 22 to the control unit 60.

The imaging unit 22 is provided so as to extend over substantially the entire width at the central portion of a lower surface of the probe 20. The imaging unit 22 can be an echographic device that includes a transducer that generates an ultrasound wave and obtain the cross-sectional image of the inside of the human body by detecting a reflected wave of the ultrasound wave. In the present embodiment, the cross-sectional image substantially orthogonal to an axial direction of the blood vessel is acquired, and thus, the imaging unit 22 is disposed such that a length direction of the imaging unit 22 is orthogonal to a length direction of the arm H.

The transmitter 23 transmits a signal from the control unit 60 to the imaging unit 22 in order to output an ultrasound wave from the imaging unit 22. The receiver 24 transmits, to the control unit 60, a signal output from the imaging unit 22 receiving the reflected wave.

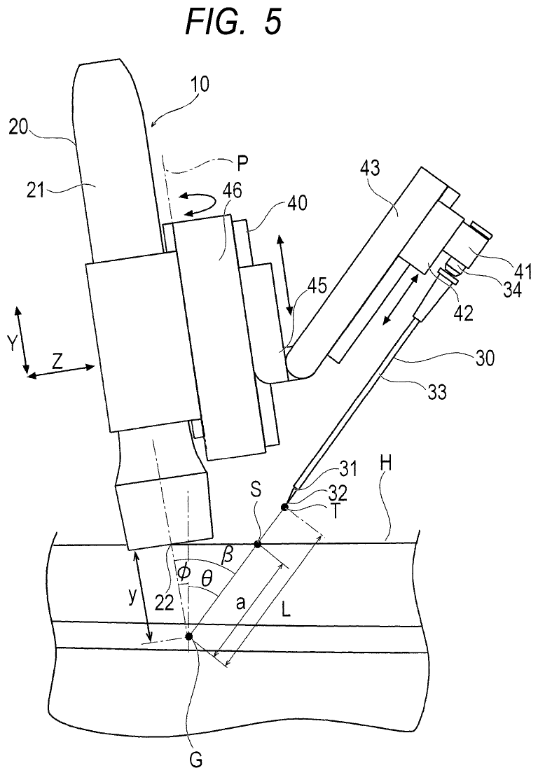

The inclination detection unit 50 is connected to the control unit 60. The inclination detection unit 50 can be, for example, a gyro sensor, and can detect an inclination of the probe 20. A reference of the inclination is a perpendicular direction orthogonal to the horizontal direction. Since an upper surface of the arm H when performing the puncture is set along the horizontal direction, an inclination of the vascular puncture system 10 with respect to a perpendicular line of the skin surface can be detected by detecting the inclination with respect to the perpendicular direction by the inclination detection unit 50. In the present example, it is assumed that the inclination detection unit 50 detects that the vascular puncture system 10 is inclined at an angle of φ as illustrated in FIG. 5. Note that the inclination detection unit 50 is not limited to the gyro sensor, and may be, for example, a camera that captures an image of the skin surface of the arm H. In this case, the control unit 60 can detect the inclination φ of the probe 20 from a result of the image capturing by the inclination detection unit 50 using a technique such as machine learning or deep learning. In addition, the inclination detection unit 50 is not necessarily provided.

As illustrated in FIG. 1, the puncture unit 30 includes a hollow inner needle 31 made of metal and having a sharp needle tip 32 formed at a distal end, and a flexible tubular outer tube 33 disposed so as to cover an outer peripheral surface of the inner needle 31. The needle tip 32 protrudes from the outer tube 33 in a state where the outer tube 33 covers the outer side of the inner needle 31. A tubular inner needle hub 34 is fixed to a proximal end portion of the inner needle 31. The outer tube 33 is not necessarily provided in the puncture unit 30.

As illustrated in FIGS. 1 and 2, the drive unit 40 includes: a holding portion 41 that holds the inner needle hub 34; a first linear movement portion 42 that linearly moves the holding portion 41; an inclination portion 43 that inclines the holding portion 41; a second linear movement portion 45 that moves the inclination portion 43 in a length direction of the probe 20; and a rotation portion 46 that rotates the second linear movement portion 45 about a predetermined rotation axis P.

The holding portion 41 can detachably hold the inner needle hub 34. The holding portion 41 can be, for example, a clamp that can perform holding so as to sandwich the inner needle hub 34.

The first linear movement portion 42 can linearly move the holding portion 41 holding the inner needle hub 34 of the puncture unit 30 forward and backward along an extending direction (puncture direction) of the inner needle 31. The first linear movement portion 42 can be used to adjust a position of the inner needle 31 and puncture the blood vessel with the inner needle 31. The first linear movement portion 42 can include, for example, a rotational drive source such as a motor whose driving can be controlled by the control unit 60, and a structure (for example, a feed screw mechanism) that converts a rotational motion of the rotational drive source into a linear motion.

The inclination portion 43 is used to change a puncture angle of the inner needle 31 with respect to a surface of a skin of the patient. The inclination portion 43 includes a hinge 44 whose angle can be changed, and a rotational drive source such as a motor whose driving can be controlled by the control unit 60 in order to change the angle of the hinge 44.

The second linear movement portion 45 is used to bring the puncture unit 30 close to (i.e., towards) or away from the skin of the patient. The second linear movement portion 45 can linearly move the inclination portion 43 forward and backward along an extending direction of the probe 20. The second linear movement portion 45 can include, for example, a rotational drive source such as a motor whose driving can be controlled by the control unit 60, and a structure (for example, a feed screw mechanism) that converts a rotational motion of the rotational drive source into a linear motion.

The rotation portion 46 is used to change a direction of the inner needle 31 when the second linear movement portion 45 is viewed substantially perpendicularly to the surface of the skin of the patient. The rotation portion 46 can rotate the inclination portion 43 about the rotation axis P parallel to the length direction of the probe 20. The rotation portion 46 can include, for example, a rotational drive source such as a motor whose driving can be controlled by the control unit 60.

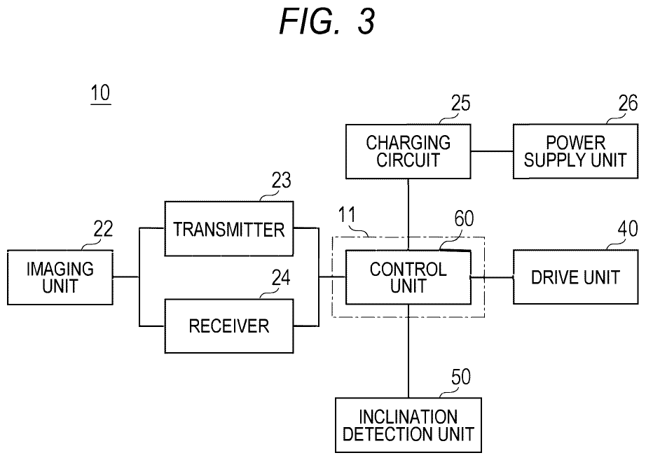

As illustrated in FIG. 3, the control unit 60 transmits a signal to the imaging unit 22 via the transmitter 23 and causes the imaging unit 22 to output an ultrasound wave. In addition, the control unit 60 can form a cross-sectional image from a signal obtained from the imaging unit 22 via the receiver 24. Further, the control unit 60 can cause a display device such as a monitor to display the obtained cross-sectional image. Further, the control unit 60 can perform arithmetic processing such as image analysis from information on the cross-sectional image to control the operation of the drive unit 40. The control unit 60 can include, as physical configurations, a storage circuit and an arithmetic circuit. The storage circuit can store programs and various parameters. The arithmetic circuit can perform arithmetic processing.

The control unit 60 can be connected to a power supply unit 26 including a rechargeable battery via a charging circuit 25. In addition, the control unit 60 is connected to the inclination detection unit 50. The control unit 60 may be disposed in the probe 20 or the drive unit 40, or may be configured separately from the probe 20 or the drive unit 40.

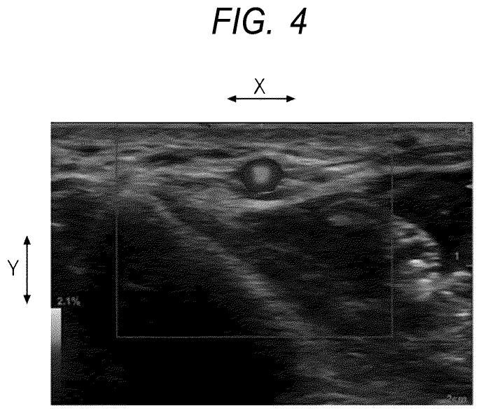

The control unit 60 acquires a cross-sectional image as illustrated in FIG. 4 from the imaging unit 22. It is assumed that a lateral direction in the cross-sectional image, that is, a width direction of the arm H is an X direction, a longitudinal direction in the cross-sectional image, that is, a depth direction of the arm H is a Y direction, and a direction orthogonal to the paper surface of the cross-sectional image, that is, the length direction of the arm H is a Z direction. Coordinates of an upper left point in the cross-sectional image are set as a start point (0, 0, 0).

The control unit 60 can identify a position of the blood vessel in the image by performing image analysis of the acquired cross-sectional image. In addition, the control unit 60 can identify the presence or absence or magnitude of pulsation, a blood flow direction, a thickness of a media, and a level of an elasticity of the blood vessel by the image analysis, and determine whether or not the blood vessel is an artery based on the result. Further, the control unit 60 can control the operation of the drive unit 40. Note that the analysis and control in the control unit 60 will be described in detail later.

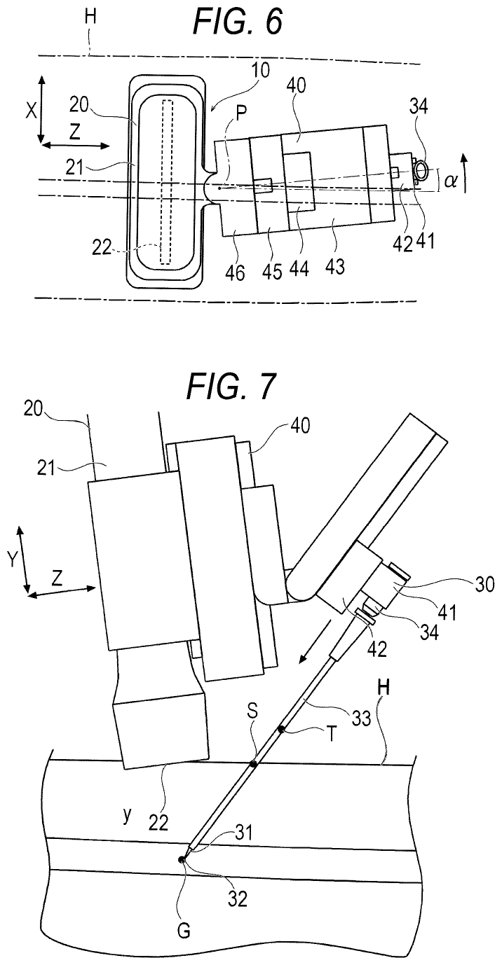

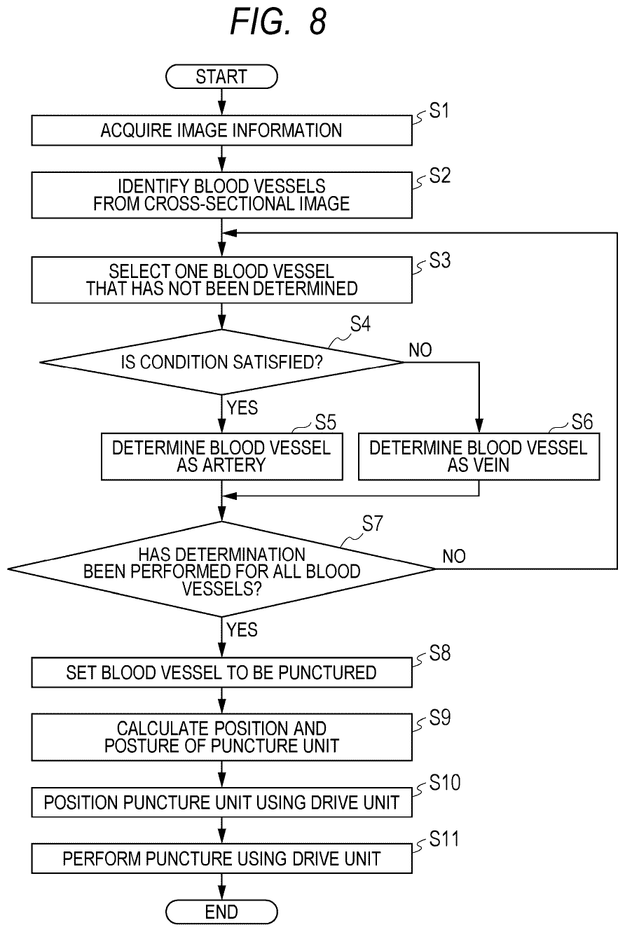

Next, a method of identifying and puncturing an artery using the vascular puncture system 10 will be described with reference to a flowchart illustrated in FIG. 8. As illustrated in FIGS. 1 and 2, the vascular puncture system 10 is used in contact with the skin surface as illustrated in FIGS. 5 and 6.

The control unit 60 acquires image information from the imaging unit 22 via the receiver 24 (S1). The control unit 60 forms a cross-sectional image from the image information. The control unit 60 can display the cross-sectional image on the monitor such that the operator can visually recognize the cross-sectional image.

The control unit 60 performs image analysis on the obtained cross-sectional image to identify blood vessels in the image (S2). In order to identify the blood vessels in the image, the control unit 60 can prepare a large number of images of the same type and use a technique such as machine learning or deep learning. In addition, it is also possible to detect a region with blood flow by the Doppler method in the imaging unit 22 and recognize the region as a region of the blood vessel. The number of blood vessels to be identified is not particularly limited.

Next, the control unit 60 selects one of the identified blood vessels (S3) and determines whether a condition indicating that the blood vessel is an artery is satisfied (S4). In order to determine that the blood vessel is an artery, information on the presence or absence or magnitude of pulsation, a blood flow direction, a thickness of a media, and a magnitude of elasticity, which are detected from the cross-sectional image, is used in the present embodiment.

First, a method of determining whether a blood vessel is an artery or a vein from the presence or absence or magnitude of pulsation will be described. Pulsation (heartbeat) is observed in arteries, but is rarely observed in veins. Therefore, in a case where the pulsation can be detected by the image analysis from the cross-sectional image of the identified blood vessel, the control unit 60 can determine that the blood vessel is an artery. The pulsation can be detected by, for example, a temporal change in the cross-sectional image of the blood vessel or image analysis using the Doppler method from the cross-sectional image of the identified blood vessel. In a case which the magnitude of the pulsation can be detected, the control unit 60 can determine that the blood vessel is an artery when the magnitude of the pulsation is equal to or greater than a predetermined threshold (or exceeds the threshold).

Next, a method of determining whether a blood vessel is an artery or a vein from a blood flow direction will be described. In the arm H, arterial blood flows towards the periphery and venous blood flows towards the center. That is, a blood flow direction in arteries is opposite to a blood flow direction in veins. The control unit 60 can detect the blood flow direction from the cross-sectional image of the identified blood vessel by image analysis using the Doppler method. In the image analysis using the Doppler method, a relative speed between an ultrasound wave and blood flow is measured in a state where a direction in which the imaging unit 22 is oriented is inclined from a direction perpendicular to an extending direction of the blood vessel. Since a relative speed between the ultrasound wave and blood flow in an artery is different from a relative speed between the ultrasound wave and blood flow in a vein, the control unit 60 can determine whether the blood vessel is the artery or the vein.

In a case where there is one artery and one vein in the cross-sectional image, it is also possible to relatively compare blood flow directions observed in the two blood vessels and determine one of the blood vessels as an artery.

Next, a method of determining whether a blood vessel is an artery or a vein from a thickness of a media will be described. A blood vessel includes an intima (endothelial cells), a media (smooth muscle) and an adventitia from the inside to the outside. An artery has a developed media since the internal pressure increases due to pulsation from the heart. The control unit 60 can detect the thickness of the media by the image analysis from the cross-sectional image of the identified blood vessel. The media is displayed in a spot pattern with low brightness in an ultrasound cross-sectional image. On the other hand, the intima and the adventitia are displayed with high brightness, and thus, the control unit 60 can identify a portion that has characteristics peculiar (i.e., particular) to the media existing between the intima and the adventitia from the cross-sectional image by the image analysis, and can identify the thickness of the media. In a case which the thickness of the media is equal to or larger than a predetermined threshold (or exceeds the threshold), the control unit 60 can determine that the blood vessel is an artery. Alternatively, in a case where there is one artery and one vein in the cross-sectional image, it is also possible to relatively compare thicknesses of media in the respective blood vessels and determine a blood vessel having a larger thickness of the media as an artery.

Next, a method of determining whether a blood vessel is an artery or a vein from an elasticity of a blood vessel will be described. A media of an artery is more developed than a media of a vein, and thus, has a higher elasticity. Therefore, the control unit 60 can detect a recovery speed of displacement after the operator pushes the skin with the imaging unit 22, squashes the skin once to make the blood vessel flat, and then releases the skin. The control unit 60 can use the recovery speed of displacement as an index indicating the elasticity (hardness). In a case where the elasticity is equal to or higher than a predetermined threshold (or exceeds the threshold), the control unit 60 can determine that the blood vessel is an artery. Alternatively, in a case where there is one artery and one vein in the cross-sectional image, it is also possible to relatively compare elasticities in the respective blood vessels and determine a blood vessel having a higher elasticity as an artery. Note that the probe 20 may be provided with a force sensor that detects a pushing force by the imaging unit 22, and the control unit 60 may analyze a measurement result obtained by the force sensor to detect the pushing force. Alternatively, the control unit 60 can create a color map of a hardness of tissue by elastography from a detection result of the imaging unit 22. Based on this, the control unit 60 can also determine a hardness of the blood vessel. Further, the hardness of the blood vessel can also be evaluated from the cross-sectional image acquired by the imaging unit 22. The control unit 60 may combine the respective methods for detecting the hardness.

When a condition indicating that the first blood vessel is an artery is satisfied, the control unit 60 determines that the blood vessel is an artery (S5), and determines that the blood vessel is a vein when the condition is not satisfied (S6). The control unit 60 determines whether a blood vessel is an artery or a vein as described above for all the identified blood vessels. When determining that the determination has been completed for all the blood vessels (S7), the control unit 60 sets an artery to be punctured from the determination result. In the present embodiment, when it is determined that a blood vessel is an artery in at least one of the determination based on the presence or absence or magnitude of pulsation, the determination based on the blood flow direction, the determination based on the thickness of the media, or the determination based on the magnitude of the elasticity described above, it is determined that the blood vessel is the artery in a comprehensive manner. The artery determination method is not limited thereto, and for example, a determination method other than the determination based on the presence or absence or magnitude of pulsation, the determination based on the blood flow direction, the determination based on the thickness of the media, and the determination based on the magnitude of the elasticity described above may be added. Alternatively, only one, two, or three of the determination based on the presence or absence or magnitude of pulsation, the determination based on the blood flow direction, the determination based on the thickness of the media, or the determination based on the magnitude of the elasticity described above may be used for the determination. Alternatively, it may be determined that the blood vessel is the artery when the condition is satisfied in a predetermined number or more (for example, two or more, three or more, or four or more) of the determination based on the presence or absence or magnitude of pulsation, the determination based on the blood flow direction, the determination based on the thickness of the media, and the determination based on the magnitude of the elasticity described above. Alternatively, the determination based on the presence or absence or magnitude of pulsation, the determination based on the blood flow direction, the determination based on the thickness of the media, and the determination based on the magnitude of the elasticity described above may be prioritized, and the determination may be performed in descending order of priority. Then, in a case where it can be determined that a blood vessel is an artery in each determination, the blood vessel can be comprehensively determined as the artery and the comprehensive determination can be ended. In a case where it cannot be determined that the blood vessel is an artery in each determination, the next determination in priority can be performed.

In a case where a plurality of blood vessels are determined to be arteries, the control unit 60 selects and sets an artery to be punctured according to a preset condition (S8). For example, the control unit 60 can detect and compare blood vessel diameters (inner diameters or outer diameters) of the plurality of arteries from the cross-sectional image, and select an artery having the largest blood vessel diameter as the artery to be punctured. In a case where an artery having a large blood vessel diameter is selected, puncture is facilitated, and a medical device such as a sheath, a catheter, or a guide wire can be rather easily inserted from a hole formed by the puncture. In a case where an artery having a small blood vessel diameter is selected, hemostasis from a hole, formed by puncture, can be facilitated. In a case where it is determined that only one blood vessel is an artery, the control unit 60 can set the artery as the artery to be punctured. In a setting in which the number of identified arteries is not two or more, the control unit 60 can set a blood vessel determined to be an artery directly as the artery to be punctured.

The control unit 60 sets a position G of the center of gravity of the region recognized as the selected blood vessel in the image as the position of the blood vessel. Coordinates of the detected position G of the center of gravity of the blood vessel are defined as (x, y, 0). In addition, the control unit 60 can also identify a position and a thickness of a calcified portion of the selected blood vessel from the cross-sectional image. The calcified portion can be identified by using a machine learning or deep learning technique, for example, from a difference in brightness value or acoustic shadow in the cross-sectional image.

Next, the control unit 60 calculates a position (coordinates) and a posture (angle) of the puncture unit 30 desirable for the puncture (S9). In the present embodiment, the control unit 60 calculates, for example, a preparation position T, a puncture angle θ, and a rotation angle α. The preparation position T is a position of the needle tip 32 immediately before puncture. The puncture angle θ is an angle at which the inner needle 31 at the time of puncture is inclined with respect to a perpendicular line of the skin surface. The rotation angle α is an angle at which the inner needle 31 at the time of puncture is inclined with respect to the Z direction when the surface of the arm H is viewed from a direction of the perpendicular line. The puncture angle θ can also be, for example, a preset angle (for example, 30 degrees). The rotation angle α is set within a range in which the needle tip 32 of the inner needle 31 can reach the inside of an artery. The preparation position T is set at a certain height from the skin surface. The preparation position T is a position where the inner needle 31 can reach the inside of the artery on the cross-sectional image by being caused to protrude along the extending direction (puncture direction). In addition, the control unit 60 may also calculate a desirable puncture speed for puncturing (a movement speed of the puncture unit 30 in the puncture direction at the time of puncture).

In addition, the control unit 60 may calculate the preparation position T, the puncture angle θ, the puncture direction α, and the puncture speed in accordance with properties of the artery such that the inner needle 31 can puncture the artery through a desired path. For example, the control unit 60 can calculate the preparation position T, the puncture angle θ, the puncture direction α, and the puncture speed such that the inner needle 31 can pass through a path avoiding the calcified portion identified from the cross-sectional image of the blood vessel. In addition, the control unit 60 may calculate the preparation position T, the puncture angle θ, the puncture direction α, the puncture speed, and the like such that the inner needle 31 can pass through a desired path according to the pulsation of the artery or the thickness of the media.

The control unit 60 first acquires a cross-sectional image from the imaging unit 22. In the cross-sectional image, the Y direction is inclined at the angle of φ with respect to the perpendicular line of the skin surface. In addition, the control unit 60 acquires the inclination φ of the vascular puncture system 10 by the inclination detection unit 50. The control unit 60 sets an upper left end position of the acquired cross-sectional image as a start point (0, 0, 0). With this start point as a reference, the control unit 60 detects the position G of the center of gravity of each blood vessel from the cross-sectional image.

For example, coordinates of the position G of the center of gravity of one detected blood vessel are set to (x, y, 0), and the rotation angle α is simply set to 0 degrees. A coordinate y1 in the Y direction of a puncture position S on the skin surface can be calculated by y1=y−a·cos(φ+θ) as illustrated in FIG. 5. A coordinate z1 in the Z direction of the puncture position S can be calculated by z1=a·sin(φ+θ). In addition, a puncture depth a is calculated by a=y·cos φ/cos θ. As a result, coordinates (x, y1, z1) of the puncture position S and the puncture depth a are defined.

A distance L from the preparation position T where the needle tip 32 is disposed to the position G of the center of gravity is set to a value longer than the puncture depth a. An angle β between a plane of the cross-sectional image and the puncture direction is obtained by β=θ+φ, and coordinates of the preparation position T can be identified by defining a puncture distance L from the position G of the center of gravity to the puncture position S, the rotation angle α, and the angle β. When the coordinates of the preparation position T are (x, y2, z2) and the rotation angle α is 0 degrees, the coordinate y2 in the Y direction can be calculated by y2=y−L·cos(φ+θ). The coordinate z2 in the Z direction can be calculated by z2=L·sin(φ+θ).

Next, the control unit 60 controls and drives at least one of the first linear movement portion 42, the second linear movement portion 45, the inclination portion 43, or the rotation portion 46 such that the inner needle 31 satisfies the puncture distance L, the rotation angle α, and the angle β. As a result, the puncture unit 30 is positioned at the desired position with the desired posture (S10). At this time, the needle tip 32 of the inner needle 31 is disposed at the preparation position T.

Next, the control unit 60 receives an instruction to start the puncture from the operator by an input means such as a switch, a keyboard, or a mouse connected to the control unit 60. In response to this instruction, the control unit 60 drives the first linear movement portion 42 (S11). As a result, as illustrated in FIG. 7, the needle tip 32 moves from the preparation position T to the inside of the artery (for example, the position G of the center of gravity) through the puncture position S. As a result, the inner needle 31 punctures the blood vessel, and the needle tip 32 reaches the inside of the blood vessel. At this time, the distal end of the outer tube 33 reaches the inside of the blood vessel together with the inner needle 31. The needle tip 32 reaches on the cross-sectional image and is observed on the monitor.

Next, the operator holds a position of the outer tube 33 so as not to move with respect to the skin. Thereafter, the control unit 60 drives the first linear movement portion 42 to retract the inner needle 31 in response to an instruction from the operator. As a result, the inner needle 31 can be removed from the blood vessel and the skin with the outer tube 33 being left in the state of being inserted into the artery.

As described above, the vascular puncture device 11 according to the present embodiment is the vascular puncture device 11 that punctures a blood vessel using the imaging unit 22 that comes into contact with a skin surface and acquires a cross-sectional image of a human body, the puncture unit 30 including the sharp needle tip 32, and the drive unit 40 that moves the puncture unit 30, and includes the control unit 60 that is capable of receiving information on the cross-sectional image and controls an operation of the drive unit 40. The control unit 60 identifies at least one blood vessel from the information on the cross-sectional image, determines whether the blood vessel is an artery or a vein from the information on the cross-sectional image of the identified blood vessel, sets a blood vessel to be punctured from among a plurality of the identified blood vessels, and controls the drive unit 40 to move the puncture unit 30 to puncture the blood vessel set to be punctured with the puncture unit 30.

The vascular puncture device 11 configured as described above can automatically detect and puncture a blood vessel, and thus, can suppress erroneous puncture of an unintended blood vessel and puncture the blood vessel with relatively high positional accuracy regardless of skill of an operator.

In addition, in a case where pulsation of the identified blood vessel can be detected from the cross-sectional image, or in a case where the detected pulsation is equal to or larger than a threshold or exceeds the threshold, the control unit 60 identifies that the blood vessel is the artery. As a result, the vascular puncture device 11 can automatically identify whether the blood vessel is the artery or the vein by detecting the pulsation existing in the artery from the cross-sectional image.

In addition, the control unit 60 detects a blood flow direction of the identified blood vessel from the cross-sectional image, and determines whether the blood vessel is the artery or the vein based on the blood flow direction. As a result, the vascular puncture device 11 can automatically and relatively easily determine whether the blood vessel is the artery or the vein by utilizing a fact that blood flows is in opposite directions between the artery and the vein.

In addition, the control unit 60 detects a thickness of a media of the identified blood vessel from the cross-sectional image, and determines whether the blood vessel is the artery or the vein based on the thickness of the media. As a result, the vascular puncture device 11 can automatically and relatively easily determine whether or not the blood vessel is the artery by utilizing a fact that the thickness of the media in the artery is larger than that in the vein.

In addition, the control unit 60 detects an elasticity of the identified blood vessel from the cross-sectional image, and determines whether the blood vessel is the artery or the vein based on the elasticity. As a result, the vascular puncture device 11 can automatically and rather easily determine whether the blood vessel is the artery or the vein by utilizing a fact that the elasticity is higher in the artery than in the vein.

In addition, the control unit 60 determines whether the blood vessel is the artery or the vein based on whether or not at least one of presence or absence or a magnitude of pulsation, a blood flow direction, a thickness of a media, or a magnitude of elasticity satisfies a condition indicating that the blood vessel is the artery from the cross-sectional image of the identified blood vessel. As a result, the vascular puncture device 11 determines whether the blood vessel is the artery or the vein by utilizing a lot of pieces of information, and thus, can relatively easily determine the artery with rather high accuracy.

In addition, the control unit 60 adjusts a position (a position of the needle tip 32) and an angle (the rotation angle α and/or the angle β) of the puncture unit 30 by the drive unit 40 such that the puncture unit 30 can puncture the blood vessel determined to be the artery in the cross-sectional image. As a result, the vascular puncture device 11 can relatively easily puncture a target artery with the puncture unit 30 with rather high accuracy while confirming the puncture unit 30 in the cross-sectional image.

In addition, the control unit 60 detects pulsation of the blood vessel determined to be the artery from the cross-sectional image, and adjusts at least one of a position (a position of the needle tip 32) or an angle (the rotation angle α and/or the angle β) of the puncture unit 30 before puncture, or a puncture speed during the puncture according to a pulsation rate. As a result, the vascular puncture device 11 can puncture the blood vessel through a desired path according to a property of the artery that changes due to the pulsation.

In addition, the control unit 60 detects a thickness of a media of the blood vessel determined to be the artery or the vein from the cross-sectional image, and adjusts at least one of a position (a position of the needle tip 32) or an angle (the rotation angle α and/or the angle β) of the puncture unit 30 before puncture, or a puncture speed during the puncture according to the thickness of the media. As a result, the vascular puncture device 11 can puncture the blood vessel through a desired path according to the thickness of the media.

In addition, the control unit 60 detects a thickness and/or a position of a calcified portion present in the blood vessel determined to be the artery or the vein from the cross-sectional image, and adjusts at least one of a position (a position of the needle tip 32) or an angle (the rotation angle α and/or the angle β) of the puncture unit 30 before puncture, or a puncture speed during the puncture according to the thickness and/or the position of the calcified portion. As a result, the vascular puncture device 11 can puncture the blood vessel through a desired path according to the thickness and/or the position of the calcified portion.

In addition, when determining whether the blood vessel is the artery or the vein, the control unit 60 may determine whether the blood vessel is the artery or the vein by a machine-learned model from the information on the cross-sectional image of the identified blood vessel. As a result, the control unit 60 can perform relatively highly accurate determination based on a plurality of pieces of stacked data.

The vascular puncture system 10 according to the present embodiment is the vascular puncture system 10 including: the imaging unit 22 that comes into contact with a skin surface and acquires a cross-sectional image of a human body, the puncture unit 30 including the sharp needle tip 32; the drive unit 40 that moves the puncture unit 30; and the control unit 60 that is capable of receiving information on the cross-sectional image and controls an operation of the drive unit 40. The control unit 60 identifies at least one blood vessel from the information on the cross-sectional image, determines whether the blood vessel is an artery or a vein from the information on the cross-sectional image of the identified blood vessel, sets a blood vessel to be punctured from among a plurality of the identified blood vessels, and controls the drive unit 40 to move the puncture unit 30 to puncture the blood vessel set to be punctured with the puncture unit 30.

The vascular puncture system 10 configured as described above can automatically detect and puncture a blood vessel, and thus, can suppress erroneous puncture of an unintended blood vessel and puncture the blood vessel with relatively high positional accuracy regardless of skill of an operator.

Note that the present disclosure is not limited to the above-described embodiment, and various modifications can be made by those skilled in the art within the technical idea of the present disclosure. For example, in the above-described embodiment, the drive unit 40 includes four movable portions (the first linear movement portion 42, the second linear movement portion 45, the rotation portion 46, and the inclination portion 43), but the number of movable portions may be five or more or three or less.

In addition, the position G of the center of gravity of the blood vessel to be punctured is detected from the cross-sectional image, and the puncture position S on the skin surface and the preparation position T are calculated from the position G of the center of gravity in the present embodiment. However, the puncture position S and the preparation position T may be calculated by detecting a position other than the position G of the center of gravity of a blood vessel to be punctured. For example, the control unit 60 may detect a position in an inner surface of a blood vessel to be punctured located between the blood vessel and the imaging unit 22 or in a membrane of the blood vessel from a cross-sectional image, and calculate the puncture position S and the preparation position T based on coordinates of the position. In addition, the control unit 60 may detect the position in the inner surface of the blood vessel to be punctured located between the blood vessel and the imaging unit 22 or in the membrane of the blood vessel from the cross-sectional image, and calculate the puncture position S and the preparation position T from coordinates of a position separated from this position by a certain distance.

In addition, after it is determined whether the blood vessel is the artery or the vein from the information on the cross-sectional image of the identified blood vessel, the blood vessel to be punctured may be selected from the blood vessel determined to be the vein instead of selecting the blood vessel to be punctured from the blood vessel determined to be the artery. In this case, a parameter for determining the vein is opposite to a parameter used in the case of determining the artery.

In addition, the vascular puncture device 11 or the vascular puncture system 10 may have a function of displaying a blood vessel determined to be punctured or a medical device adapted to a punctured blood vessel. An operator inserts, for example, a sheath along the outer tube 33 after puncturing the blood vessel with the puncture unit 30 and removing the inner needle 31. An outer diameter of the sheath is preferably equal to or smaller than an inner diameter of the blood vessel into which the sheath is to be inserted. This is because when the outer diameter of the sheath is equal to or larger than the inner diameter of the blood vessel, complications are likely to be caused by inserting the sheath into the blood vessel. As an example of a method of calculating the inner diameter of the blood vessel, a length of a diagonal line passing through a center of gravity of an inner peripheral surface of the identified blood vessel (artery or vein) is acquired for the entire circumference at predetermined angle intervals (for example, in the interval of 1 degree), and an average value of the length of the diagonal line passing through the center of gravity of the inner peripheral surface of the identified blood vessel (artery or vein) can be set as the inner diameter of the blood vessel. Alternatively, the inner diameter of the blood vessel may be calculated back from an area of the inside of the inner peripheral surface of the blood vessel. When an inner diameter of a blood vessel of an artery, it is preferable to detect the inner diameter of the blood vessel at a certain timing since the artery pulsates. In addition, the certain timing is preferably a timing when the blood vessel contracts most. Since the minimum inner diameter of the inner diameter of the blood vessel is larger than the outer diameter of the medical device to be inserted, the occurrence of complications can be reduced. After calculating the inner diameter of the blood vessel, the control unit 60 can display the outer diameter and a product type of the medical device adapted to the calculated inner diameter of the blood vessel on a display device such as a monitor together with the cross-sectional image. The control unit 60 may identify at least one of an optimal outer diameter, length, or product type of the inner needle 31 from information on a blood vessel determined to be punctured, past statistical information, and the like, display the same on a display device such as a monitor together with a cross-sectional image to present the same to the operator.

In addition, the drive unit 40 may be a robot arm.

The detailed description above describes embodiments of a vascular puncture device and a vascular puncture system capable of detecting and puncturing a position of a blood vessel from an image acquired by an echographic device. The invention is not limited, however, to the precise embodiments and variations described. Various changes, modifications and equivalents may occur to one skilled in the art without departing from the spirit and scope of the invention as defined in the accompanying claims. It is expressly intended that all such changes, modifications and equivalents which fall within the scope of the claims are embraced by the claims.

What is claimed is:

1. A vascular puncture device configured to puncture a blood vessel, the vascular puncture device comprising:
an imaging unit configured to come into contact with a skin surface and acquire a cross-sectional image of a human body;
a puncture unit including a needle tip;
a drive unit configured to move the puncture unit;
a control unit configured to receive information on the cross-sectional image and control an operation of the drive unit; and
wherein the control unit is configured to:
identify at least one blood vessel from information on the cross-sectional image;
determine whether the identified at least one blood vessel is an artery or a vein from information on the cross-sectional image of the identified at least one blood vessel;
set a blood vessel to be punctured from among the identified at least one blood vessel; and
control the drive unit to move the puncture unit to puncture the blood vessel set to be punctured with the puncture unit.

2. The vascular puncture device according to claim 1, wherein the control unit is configured to determine that the identified at least one blood vessel is the artery in a case where pulsation of the identified at least one blood vessel is detectable from the cross-sectional image, or in a case where the detected pulsation is equal to or larger than a threshold or exceeds the threshold.

3. The vascular puncture device according to claim 1, wherein the control unit is configured to detect a blood flow direction of the identified at least one blood vessel from the cross-sectional image, and determine whether the identified at least one blood vessel is the artery or the vein based on the blood flow direction.

4. The vascular puncture device according to claim 1, wherein the control unit is configured to detect a thickness of a media of the identified at least one blood vessel from the cross-sectional image, and determine whether the identified at least one blood vessel is the artery or the vein based on the thickness of the detect media of the identified at least one blood vessel.

5. The vascular puncture device according to claim 1, wherein the control unit is configured to detect an elasticity of the identified blood vessel from the cross-sectional image, and determine whether the identified at least one blood vessel is the artery or the vein based on the detected elasticity of the identified at least one least one blood vessel.

6. The vascular puncture device according to claim 1, wherein the control unit is configured to determine whether the identified at least one blood vessel is the artery or the vein based on whether or not at least one of presence or absence or a magnitude of pulsation, a blood flow direction, a thickness of a media, or a magnitude of elasticity satisfies a condition indicating that the identified at least one blood vessel is the artery from the cross-sectional image of the identified at least one blood vessel.

7. The vascular puncture device according to claim 1, wherein the control unit is configured to adjust a position and an angle of the puncture unit by the drive unit to enable the puncture unit to puncture the identified at least one blood vessel determined to be the artery in the cross-sectional image.

8. The vascular puncture device according to claim 1, wherein the control unit is configured to detect a pulsation of the identified at least one blood vessel determined to be the artery from the cross-sectional image, and adjust at least one of a position or an angle of the puncture unit before puncture or a puncture speed during the puncture according to a pulsation rate.

9. The vascular puncture device according to claim 1, wherein the control unit configured to detect a thickness of a media of the identified at least one blood vessel determined to be the artery or the vein from the cross-sectional image, and adjust at least one of a position or an angle of the puncture unit before puncture or a puncture speed during the puncture according to the detected thickness of the media of the identified at least one blood vessel.

10. The vascular puncture device according to claim 1, wherein the control unit is configured to detect a thickness and/or a position of a calcified portion present in the identified at least one blood vessel determined to be the artery or the vein from the cross-sectional image, and adjust at least one of a position or an angle of the puncture unit before puncture or a puncture speed during the puncture according to the thickness and/or the position of the detected calcified portion of the identified at least one blood vessel.

11. The vascular puncture device according to claim 1, wherein
when determining whether the identified at least one blood vessel is the artery or the vein, the control unit is configured to determine whether the identified at least one blood vessel is the artery or the vein by a machine-learned model from the information on the cross-sectional image of the identified at least one blood vessel.

12. A vascular puncture system comprising:
an imaging unit configured to come into contact with a skin surface and acquire a cross-sectional image of a human body;
a puncture unit including a needle tip;
a drive unit configured to move the puncture unit;
a control unit configured to receive information on the cross-sectional image and control an operation of the drive unit; and
wherein the control unit is configured to:
identify at least one blood vessel from information on the cross-sectional image;
determine whether the identified at least one blood vessel is an artery or a vein from information on the cross-sectional image of the identified at least one blood vessel;
set a blood vessel to be punctured from among of the identified at least one blood vessels; and
control the drive unit to move the puncture unit to puncture the blood vessel set to be punctured with the puncture unit.

13. A method for puncturing a blood vessel, the method comprising:
acquiring a cross-sectional image of a human body;
identifying one or more blood vessels from the acquired cross-sectional image of the human body;
determining whether the one or more blood vessels is an artery or a vein from the cross-sectional image of the human body;
setting a blood vessel to be punctured from among the one or more identified blood vessels; and
puncturing the blood vessel set to be punctured.

14. The method according to claim 13, further comprising:
determining that the one or more identified blood vessels is the artery in a case where pulsation of the blood vessel is detectable from the cross-sectional image, or in a case where the detected pulsation is equal to or larger than a threshold or exceeds the threshold.

15. The method according to claim 13, further comprising:
detecting a blood flow direction of the one or more identified blood vessels from the cross-sectional image; and
determining whether the one or more identified blood vessels is the artery or the vein based on the detected blood flow direction of the one or more identified blood vessels.

16. The method according to claim 13, further comprising:
detecting a thickness of a media of the one or more identified blood vessels from the acquired cross-sectional image; and
determining whether the one or more identified blood vessels is the artery or the vein based on the detected thickness of the media of the one or more identified blood vessels.

17. The method according to claim 13, further comprising:
detecting an elasticity of the one or more identified blood vessels from the acquired cross-sectional images; and
determining whether the one or more identified blood vessels is the artery or the vein based on the detected elasticity of the one or more identified blood vessels.

18. The method according to claim 13, further comprising:
determining whether the one or more identified blood vessels is the artery or the vein based on whether or not one or more of presence or absence or a magnitude of pulsation, a blood flow direction, a thickness of a media, or a magnitude of elasticity satisfies a condition indicating that the one or more identified blood vessels is the artery from the cross-sectional image of the one or more identified blood vessels.

19. The method according to claim 13, further comprising:
adjusting a position and an angle of a puncture unit to enable the puncture unit to puncture the one or more identified blood vessels determined to be the artery in the cross-sectional image of the one or more identified blood vessels.

20. The method according to claim 13, further comprising:
detecting a pulsation of the one or more identified blood vessels determined to be the artery from the cross-sectional image, and adjusting one or more of a position or an angle of the puncture unit before puncture or a puncture speed during the puncture according to a pulsation rate,
detecting a thickness of a media of the one or more identified blood vessels determined to be the artery or the vein from the cross-sectional image, and adjusting one or more of a position or an angle of the puncture unit before puncture or a puncture speed during the puncture according to the thickness of the media, or
detecting a thickness and/or a position of a calcified portion present in the one or more identified blood vessel determined to be the artery or the vein from the cross-sectional image, and adjusting one or more of a position or an angle of the puncture unit before puncture or a puncture speed during the puncture according to the thickness and/or the position of the calcified portion.
